(19) United States
(12) Patent Application Publication
(10) Pub. No.: US 2006/0094744 A1
(43) Pub. Date: May 4, 2006

(54) Pharmaceutical dosage forms of stable amorphous rapamycin like compounds

(76) Inventors: Cynthia A. Maryanoff, Forest Grove, PA (US); Karl Pieter Laura Stefaan Six, Hulste (BE); Roger Petrus Gerebern Vandecruys, Westerlo (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/237,301

(22) Filed: Sep. 28, 2005

Related U.S. Application Data
(60) Provisional application No. 60/614,139, filed on Sep. 29, 2004.

(51) Int. Cl. A61K 31/4745

(52) U.S. Cl. 514/291

(57) ABSTRACT

The present invention provides a pharmaceutical dosage form comprising stable amorphous rapamycin like compounds and a pharmaceutically acceptable excipient and methods of making the pharmaceutical dosage form.

### FIELD OF INVENTION

This application claims the benefit of provisional patent application 60/614,139 filed Sep. 29, 2004, which is hereby incorporated herein by reference.

The present invention relates to a pharmaceutical dosage form for delivery of stable amorphous rapamycin like compounds.

### BACKGROUND OF THE INVENTION

Rapamycin is a macrocyclic triene antibiotic produced by Streptomyces hygroscopius as disclosed in U.S. Pat. No. 3,929,992. It has been found that rapamycin among other things inhibits the proliferation of vascular smooth muscle cells in vivo. Accordingly, rapamycin may be utilized in treating intimal smooth muscle cell hyperplasia, restenosis, and vascular occlusion in a mammal, particularly following either biologically or mechanically mediated vascular injury, or under conditions that would predispose a mammal to suffering such a vascular injury. Rapamycin functions to inhibit smooth muscle cell proliferation and does not interfere with the re-endothelialization of the vascular walls.

Rapamycin reduces vascular hyperplasia by antagonizing smooth muscle proliferation in response to mitogenic signals that are released during vascular injury. Inhibition of growth factor and cytokine mediated smooth muscle proliferation at the late G1 phase of the cell cycle is believed to be the dominant mechanism of action of rapamycin. However, rapamycin is also known to prevent T-cell proliferation and differentiation when administered systemically. This is the basis for its immunosuppressive activity and its ability to prevent graft rejection.

Previously known forms of amorphous rapamycin did not have optimum shelf lives. The present invention provides amorphous rapamycin that is stable for extended period of time and is capable of being processed into pharmaceutical dosage forms, incorporated into drug delivery systems and coated on medical devices.

### SUMMARY OF THE INVENTION

The present invention provides a pharmaceutical dosage form comprising stable amorphous rapamycin like compounds and a pharmaceutically acceptable excipient.

### DETAILED DESCRIPTION OF THE INVENTION

As used herein, “rapamycin like compounds” as used herein includes rapamycin and all analogs, derivatives and conjugates that bind to FKBP12, and other immunophilins and possesses the same pharmacologic properties as rapamycin, including inhibition of the target of rapamycin (TOR). Sirolimus is a rapamycin also know as (3S,6R,7E,9R,10R,12R,14S,15E,17E,19E,21 S,23S, 26R,27R,34aS)-9, 10, 12, 13, 14,21,22,23,24,25,26,27,32,33,34, 34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4] oxaazacyclohentriacontine-1,5,11,28,29(4H,6H,31H)-pentone.

Other analogs, derivatives and conjugates that may be processed into a substantially solvent free amorphous solid include, but are not limited to, 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-y1)-rapamycin 4O—O-(2-Hydroxy)ethoxycarbonylmethyl-rapamycin, 40-O-(3-Hydroxy)propyl-rapamycin 4O—O-(6-Hydroxy)hexyl-rapamycin 40-O-[2-(2-Hydroxy)ethoxy]ethyl-rapamycin 4O—O-[(3S)-2,2-Dimethyldioxolan-3-yl]methyl-rapamycin, 40-O-[(2S)-2,3-Dihydroxyprop-1-yl]-rapamycin, 4O—O-(2-Acetoxy)ethyl-rapamycin 4O—O-(2-Nicotinoyloxy)ethyl-rapamycin, 4O—O-[2-(N-Morpholino)acetoxy]ethyl-rapamycin 4O—O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 4O—O-(2-Aminoethyl)-rapamycin, 4O—O-(2-Acetaminoethyl)-rapamycin 4O—O-(2-Nicotinamidoethyl)-rapamycin, 4O—O-(2-(N-Methyl-imidazo-2′-ylcarbethoxamido)ethyl)-rapamycin, 4O—O-(2-Ethoxycarbonylaminoethyl)-rapamycin, 40-O-(2-Tolylsulfonamidoethyl)-rapamycin, 40-O-[2-(4′,5′-Dicarboethoxy-I′,2′,3′-triazol-1′-yl)-ethyl]-rapamycin, 42-deoxy-42-(1 H-tetrazol-1-yl)-, (42S)-rapamycin (Zotarolimus) 42-[3-hydroxy-2-(hydroxymethyl)-2-methylpropanoate]rapamycin (temsirolimus), and tacrolimus.

Amorphous rapamycin like compounds, for example, sirolimus, may be prepared or processed in a manner such that it is in a stable form that may be administered in any number of ways. For example, the sirolimus may be administered orally, parenterally, intravascularly, intranasally, intrabronchially, transdermally, rectally or via a coated medical device such as a stent coated with sirolimus.

In the exemplary embodiment described herein, a crystalline rapamycin like compounds such as sirolimus may be processed into a substantially solvent free amorphous solid form. For example sirolimus may be processed into an amorphous form with a glass transition temperature of from about 91 to about 95° C. and preferably about 93° C. The glass transition is a property of amorphous materials. When an amorphous material is heated to a temperature above its glass transition temperature, the molecules comprising the material are more mobile, which in turn means that they are more active and thus more prone to reactions such as oxidation. However, when an amorphous material is maintained at a temperature below its glass transition temperature, its molecules are substantially immobilized and thus less prone to reactions such as oxidation. Accordingly, the higher the glass transition temperature for a given amorphous material, the more stable or less reactive the material is under room temperature and pressure (RTP) conditions.

Amorphous rapamycin like compounds may be prepared by mixing crystalline rapamycin like compounds with an appropriate solvent such as 2-propanol. The amount of solvent that may be used will depend on the solubility of the particular rapamycin like compounds in the specific solvent and the mixing conditions (e.g. temperature, mixing device used and the like). Preferably the amount of solvent used will be in the range of from about 2 ml to about 10 ml per gram of rapamyin like compound more preferably about 3 ml to about 5 ml per gram of rapamycin. The mixture may be heated and/or stirred to facilitate dissolution of the rapamycin like compounds (provided the heating is below the degradation temperature of the rapamycin like compound). The rapamycin like compound in solution may then be precipitated from the solution by adding and agent, which causes the rapamycin like compound to be precipitated from the solution. The preferred agent is water. The precipitate formed by this process is amorphous rapamycin like compound. The mixture of the precipitate, solvent and agent may then be used in the preparation of products and dosage forms or the precipitate may be separated from the solvent and/or agent. Suitable method of separating the precipitate from the mixture are well known to those of ordinary skill in the arts and include but not limited to drying, filtration, centrifugation and the like. It is currently preferred that the precipitate be separated from the mixture by filtration and the precipitate be washed with a suitable liquid in which the rapamycin like compound is not soluble in or has a very low solubility in under the wash conditions. The amorphous rapamycin like compound produced by this process may then be dried in a manner suitable to retain its substantially amorphous form. Preferably the amorphous rapamycin like compound will be substantially amorphous and have less than about 30 weight percent crystalline rapamycin like compound (e.g. crystalline sirolimus), more preferably less than about 10 weight percent crystalline rapamycin like compound (e.g. crystalline sirolimus), most preferably less than about 5 weight percent crystalline rapamycin like compound (e.g. crystalline sirolimus) and even more preferably less than about 1 weight percent crystalline rapamycin like compound (e.g. crystalline sirolimus). In one embodiment of the present invention the amorphous rapamycin like compound is preferably 100 weight percent amorphous rapamycin. In another embodiment of the present invention crystalline rapamycin like compounds can be added to the amorphous rapamycin like compound to vary the percentage of crystalline to amorphous rapamycin like compound.

In one exemplary embodiment of the present invention there is provided a method of preparing a substantially solvent free amorphous sirolimus with a glass transition temperature, T_{g}, of from about 91 to about 95° C. and preferably about 93° C. comprising the following steps. First, a given amount of crystalline sirolimus is dissolved in an appropriate solvent. In the exemplary embodiment, 250 mg of crystalline sirolimus is placed in a 100 ml beaker to which 4 ml of 2-propanol is added. The mixture may be slightly heated and mixed to facilitate the dissolution of the sirolimus. Next, the solution is stirred while an agent is added to the solution to precipitate the sirolimus from solution. In the exemplary embodiment, the solution is continuously stirred with a magnetic stirrer while 50 ml of water is added in order to precipitate the amorphous sirolimus. The product of this step is an amorphous precipitate. The concentration of sirolimus in solution determines the length of time required to precipitate the sirolimus from solution. Next, the amorphous precipitate is filtered and washed. In the exemplary embodiment, the amorphous precipitate is passed through a 0.45 μm pore filter under vacuum to remove the supernatant. The filtered amorphous precipitate is then washed with 100 ml of water to remove impurities. In the next and final step, the precipitate is dried. In the exemplary embodiment, the precipitate is dried for a period ranging from 18 hours to about 36 hours at a temperature of about 30° C. and under a vacuum of about 150 mBar. The result is a substantially solvent free amorphous solid form of sirolimus with a glass transition temperature of about 93° C. that may be utilized in a polymer as described herein or in any other suitable dosage form as described herein.

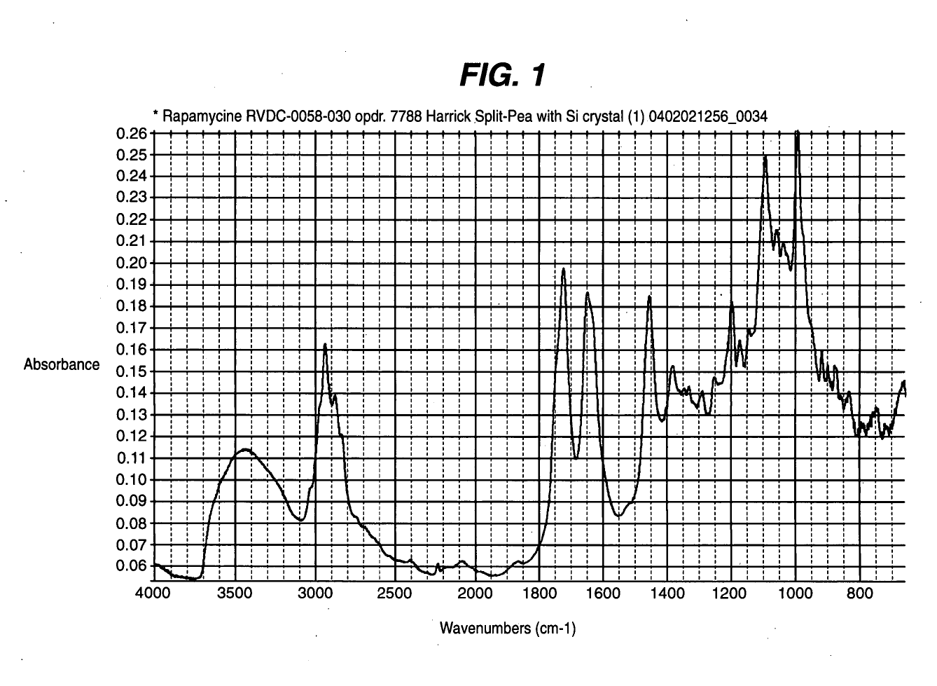

A number of tests or evaluations may be performed in order that the substantially solvent free amorphous sirolimus may be characterized. In one test, the amorphous sirolimus is analyzed utilizing a micro attenuated total reflectance (ATR) infrared spectrometer. Essentially, the purpose of this test is to determine if the amorphous sirolimus prepared by the above-described process is degraded in any significant way. Table 1, given below, contains a summary of the test parameters. FIG. 1 is the ATR-infrared spectrum of amorphous sirolimus prepared utilizing the above-described process. As illustrated in FIG. 1, the infrared spectrum of the prepared sirolimus reflects the vibrational modes of the molecular structure of sirolimus. In other words, the sirolimus was not degraded during the process.

TABLE 1
Micro Attenuated Total Reflectance Infrared Spectroscopy

| | |
|---|---|
| Number of scans: | 32 |
| Resolution: | 1 cm−1 |
| Wavelength range: | 4000 to 400 cm−1 |
| Apparatus: | NICOLET MAGNA 560 FTIR |
| | SPECTROPHOTOMETER¹ |
| Baseline correction: | yes |
| Detector: | DTGS² with KBr windows |
| Beam splitter: | Ge on KBr |
| Micro ATR accessory: | HARRICK SPLIT PEA with Si |
| | crystal |

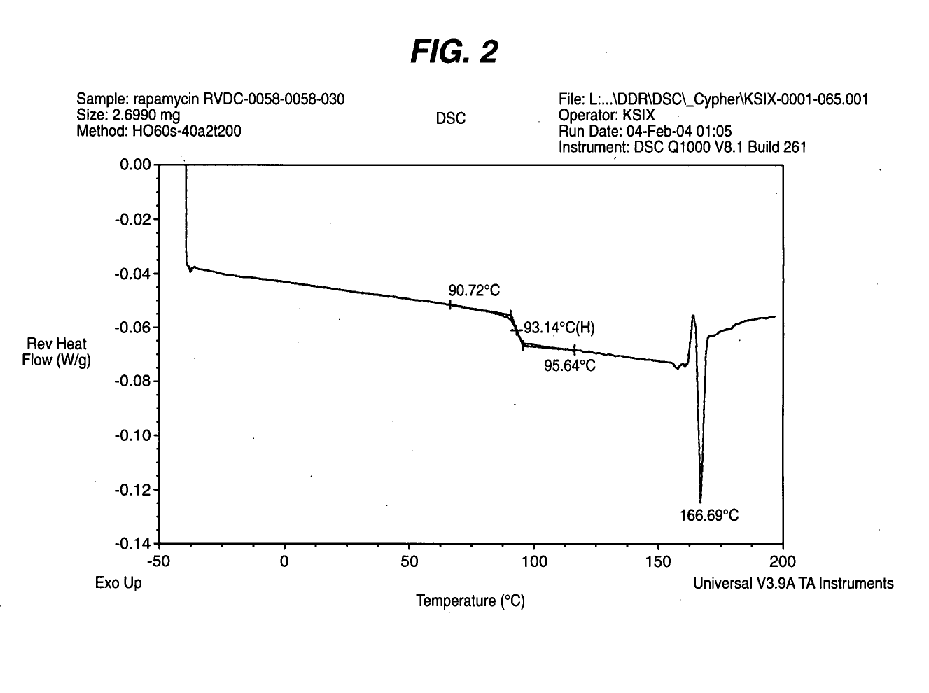

In another test, the amorphous sirolimus is analyzed utilizing differential scanning calorimetry. Essentially, the purpose of this test is to determine the glass transition temperature of the amorphous sirolimus. In this test, approximately 3 mg of amorphous sirolimus is transferred into a standard aluminum TA-Instrument sample pan and covered. The DSC curve is recorded on a TA-Instruments Q1000 MTDSC equipped with a RCS cooling unit. Table 2, given below, contains a summary of the test parameters. FIG. 2 illustrates a differential scanning calorimetry curve of amorphous sirolimus. The differential scanning calorimetry curve shows the glass transition temperature of the amorphous sirolimus to be about 93° C.

TABLE 2
Differential Scanning Calorimetry Settings
First Heating

| | |
|---|---|
| Initial temperature | 40° C. |
| Heating rate | 2° C./min |
| Final temperature | 30 ml/min |
| Nitrogen flow | 30 ml/min |
| Amplitude | 0.318° C. |
| Period | 60 s |

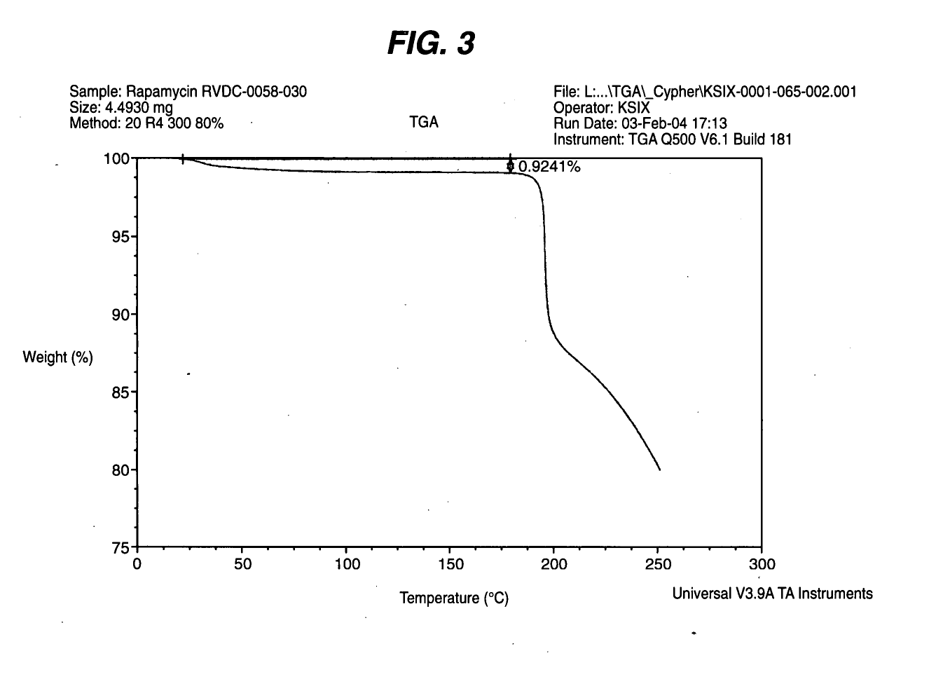

In another test, the amorphous sirolimus is analyzed utilizing a thermogravitometer. Essentially, the purpose of this test is to determine weight loss in the amorphous sirolimus. In this test, the amorphous sirolimus is transferred into an aluminum sample pan and placed in a thermogavimeter. The TG curve is recorded utilizing a TA Instruments HI-RES TGA 2950 thermogavimeter. Table 3, given below, contains a summary of the test parameters. FIG. 3 illustrates a thermogravity curve of amorphous sirolimus. As is illustrated, a loss of sample weight occurs from about 25° C. to about 160° C. This small weight loss may be due to the evaporation of absorbed water and 2-propanol. A second weight loss is observed when the compound decomposes.

TABLE 3
Thermogravimetry Parameters

| | |
|---|---|
| Initial temperature: | Room Temperature |
| Heating rate: | 20° C./min |
| Resolution factor: | 4 |
| Final condition: | 300° C. or <80[(w/w)%] |

In yet another test, the amorphous sirolimus is analyzed utilizing a gas chromatograph. Essentially, the purpose of this test is to determine the chemical composition of the sample, in particular, the residual solvent content. In this test, 15 mg of amorphous sirolimus is placed in a vial and dissolved in 2 ml of DMSO. The vial is closed and analyzed utilizing the parameters listed in Table 4, given below. The results of the test indicate that the amorphous sirolimus contains 77 ppm of 2-propanol.

TABLE 4
Gas Chromatography

| GC system | Parameters | |
|---|---|---|
| Column | 50 m fused silica column with an ID of 0.32 mm, coated | |
| | with a chemically bonded polydimethylsiloxane (CP-SIL 5 | |
| | CB) of 5 μm film thickness. | |
| | comment: | |
| Carrier gas | Gas: nitrogen 5.5 | |
| | Gas: Hydrogen | |
| | Pi: 100 kPa | |
| | Mode: constant pressure | |
| | comment: | |
| Injector | Type: | Splitter |
| | | dynamic splitting: 0-30 ml/min |
| | | split insert: fritted |
| | | temperature: 230° C. |
| | comment: | |
| Detector | Type: FID | |
| | Temperature: 270° C. | |
| | gases: | hydrogen: 23-31 ml/min |
| | | air: 285-315 ml/min |
| | | make up: 20-25 ml/min |

| | sensitivity: range 12 (1 × 10³ pA/V) | | | | | |
|---|---|---|---|---|---|---|
| | comment: | | | | | |
| Headspace | Bath temperature: | | | 80° C. | | |
| autosampler | Loop temperature: | | | 230° C. | | |
| | Loop volume: | | | 5 ml | | |
| | Equilibration time: | | | 55 min | | |
| | Pressure time on vial: | | | 2 min | | |
| | Pressure hold time: | | | 0.2 min | | |
| | Loop fill time: | | | 1 min | | |
| | Loop equilibration time: | | | 0.2 min | | |
| | Injection time: | | | 0.5 min | | |
| | Transfer line temperature: | | | 230° C. | | |
| | Vial pressure: | | | ˜50 kPa | | |
| | Transfer line pressure: | | | ˜120 kPa | | |
| | | start | rate | end | hold | |
| Temperature | | temp. | in | temp. | time | run time |
| program | Step | in ° C. | ° C./min. | in ° C. | in min. | in min. |
| | 1 | 40 | 0 | 40 | 0.5 | 0-0.5 |
| | 2 | 40 | 5 | 165 | 0 | 0.5-25.5 |
| | 3 | 165 | 30 | 220 | 8 | 25.5-35.3 |

comment: step 3 is started approximately 2 minutes after
elution of chlorobenzene

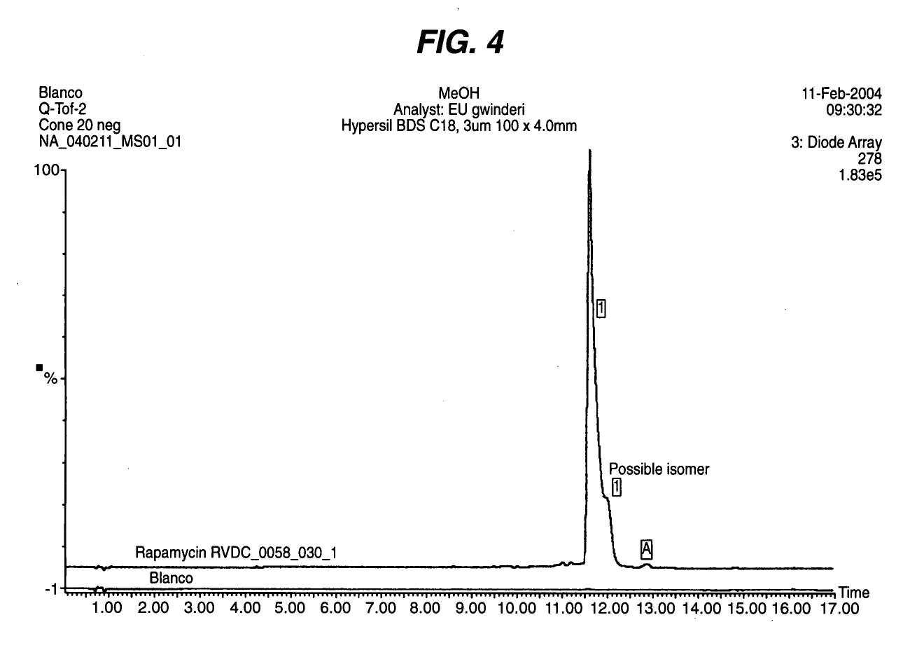

In another test the amorphous sirolimus is analyzed using High Pressure Liquid Chromatography-Mass Spectrometry (LC-MS). Table 5, given below, contains a summary of the test parameters. Essentially, the purpose of this test is to determine if the amorphous sirolimus prepared by the above-described process is degraded in any significant way. FIG. 4 is the LC-MS trace of amorphous sirolimus prepared utilizing the above-described process. LC-MS analysis on solvent free amorphous rapamycin prepared by the above-described process confirmed the formula by accurate mass. In other words, the sirolimus was not degraded during the process.

TABLE 5
High Pressure Liquid Chromatography-Mass Spectrometry (LC-MS)

| HPLC System | Parameters | | | |
|---|---|---|---|---|
| Column | Hypersil BDS - 10 cm × 4 mm I.D. and 3 μm particle | | | |
| | size | | | |
| | comment: | | | |
| Column | 30° C. | | | |
| temperature | comment: | | | |
| Flow rate | 1.2 ml/min | | | |
| | comment: | | | |
| Injection volume | 5 μl | | | |
| | comment: | | | |
| Mobile phase | preparation and composition | | | |
| | A | 0.5% ammonium acetate in water | | |
| | B | acetonitrile | | |
| | comment: | | | |
| | | time in min. | | |
| Gradient | solvent | 0 | 15 | 17 |
| | % A | 90 | 0 | 0 |
| | % B | 10 | 100 | 100 |

comment: analytical run time is 15 minutes

The term “subject” as used herein, refers to an animal, preferably a mammal, most preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The present invention further comprises pharmaceutical compositions containing one or more amorphous rapamycin like compounds with a pharmaceutically acceptable carrier. Currently the preferred amorphous rapamycin like compounds is amorphous srolimus. Pharmaceutical compositions containing one or more amorphous rapamycin like compounds described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps, geltabs and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 6 mg and may be given at a dosage of from about 0.1 mg to about 2 mg and preferably from about 0.5 mg to about 1 mg. The dosages, however, may be varied depending upon the requirement of the patients, the severity of the condition being treated and the compound being employed. The use of either daily administration or post-periodic dosing may be employed.

Preferably these pharmaceutical compositions are in unit dosage forms from such as tablets, capsules, caplets, gelcaps, geltabs, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the pharmaceutical composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, capsules, caplets and the like. This solid preformulation composition is then subdivided into unit dosage forms of the type described above containing from 0.01 mg to about 6 mg, preferably, from about 0.1 mg to about 2 mg, and more preferably from about 0.5 mg to about 1 mg of the active ingredient of the present invention. The tablets, capsules and caplets of the novel composition can be coated or otherwise compounded to provide a dosage form affording the advantage of prolonged action. For example, the tablet, capsules, or caplets can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the amorphous rapamycin like compounds of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method described in the present invention may also be carried out using a pharmaceutical composition comprising any of the compounds as defined herein and a pharmaceutically acceptable carrier. The pharmaceutical composition may contain between about 0.01 mg to about 6 mg, preferably about 0.1 mg to about 2 mg and more preferably from about 0.5 mg to about 1 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as. tablets, caplets,. capsules and the like (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, one or more of the compounds of the present invention may be administered in a single daily dose, or the total daily dosage may be administered in divided doses of two, three or four times daily. Furthermore, the amorphous rapamycin like compounds of the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the amorphous rapamycin like compound can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phophatidylcholines.

The amorphous rapamycin like compounds of this invention may be administered in any of the foregoing compositions and according to dosage regimens established in the art.

The daily dosage of the products may be varied over a wide range from 0.01 to 6 mg per adult human per day. For oral administration, the compositions are preferably provided in the form of tablets containing, 0.01, 0.05, 0.1, 0.5, 1, 2, 3, 4, 6 milligrams of the active ingredient for the symptomatic adjustment of the dosage to the patient to be treated. An effective amount of the drug is ordinarily supplied at. a dosage level of from about 0.01 mg/kg to about 1 mg/kg of body weight per day. Preferably, the range is from about 0.03 to about 0.2 mg/kg of body weight per day, most preferably, from about 0.03 to about 0.1 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

### SOLID DOSAGE FORM EXAMPLES

The following provide the preparation and evaluation of representative examples of rapamycin like compounds in solid dosage tablets.

### Prospective Example 1

The following shows the preparation and potential evaluation of a 1 mg amorphous rapamycin like compounds in oral dosage tablet containing a 100 mg sugar overcoat.

Formula

| Ingredients* | Amount | |
|---|---|---|
| Amorphous Sirolimus | 1 | mg |
| PLURONIC F68 (poloxamer 188) | 0.5 | mg |
| Sucrose | 98.940 | mg |
| Povidone | 0.510 | mg |
| Microcrystaline cellulose | 1.020 | mg |
| Water | 49.653 | mg |

*A 2% overage is included in these quantities to account for manufacturing losses.

Manufacturing Directions

1. A dispersion of less than about 400 nm particle size of amorphous sirolimus and PLURONIC F68 (poloxamer 188) is prepared according to U.S. Pat. No. 5,145,684 using a 2:1 ratio of amorphous sirolimus:PLURONIC F68. A dispersion concentration of 150 mg amorphous sirolimus/ml is used.

2. Sucrose is added and mixed until the sucrose dissolved.

3. Povidone is added and mixed until well wetted. Mixing was continued vigorously until the povidone dissolved.

4. Microcrystaline cellulose is added, and is mixed well until wetted.

5. Water is added and is mixed well.

6. The resulting solution is spray coated onto a pharmaceutically inert core portionwise and is air dried in between portions.

### Prospective Example 2

A 0.5 mg amorphous sirolimus oral dosage tablet containing a 100 mg sugar overcoat is prepared according the procedure described in Example 1. The dispersion contained a 2:1 ratio of amorphous sirolimus:PLURONIC F68 (poloxamer 188), and is used at a concentration of 150 mg amorphous sriolimus/ml. The following lists the quantities of ingredients will be used.

Formula

| Ingredients* | Amount | |
|---|---|---|
| Amorphous Sirolimus | 0.5 | mg |
| PLURONIC F68 (poloxamer 188) | 0.25 | mg |
| Sucrose | 99.705 | mg |
| Povidone | 0.510 | mg |
| Microcrystaline cellulose | 1.020 | mg |
| Water | 52.288 | mg |

*A 2% overage is included in these quantities to account for manufacturing losses.

### Prospective Example 3

A 3.0 mg amorphous sirolimus oral dosage tablet containing a 100 mg sugar overcoat is prepared according the procedure described in Example 1. The dispersion contained a 2:1 ratio of amorphous sirolimus:PLURONIC F68 (poloxamer 188), and is used at a concentration of 150 mg amorphous sirolimus/ml. The following lists the quantities of ingredients will be used.

Formula

| Ingredients* | Amount | |
|---|---|---|
| Amorphous Sirolimus | 3.0 | mg |
| PLURONIC F68 (poloxamer 188) | 1.5 | mg |
| Sucrose | 95.880 | mg |
| Povidone | 0.510 | mg |
| Microcrystaline cellulose | 1.020 | mg |
| Water | 39.113 | mg |

*A 2% overage is included in these quantities to account for manufacturing losses.

### Prospective Example 4

A 5.0 mg amorphous sirolimus oral dosage table containing a 100 mg sugar overcoat is prepared according the procedure described in Example 1. The dispersion contained a 2:1 ratio of amorphous sirolimus:PLURONIC F68 (poloxamer 188), and is used at a concentration of 150 mg amorphous sirolimus/ml. The following lists the quantities of ingredients will be used.

Formula

| Ingredients* | Amount | |
|---|---|---|
| Amorphous Sirolimus | 5.0 | mg |
| PLURONIC F68 (poloxamer 188) | 2.5 | mg |
| Sucrose | 92.820 | mg |
| Povidone | 0.510 | mg |
| Microcrystaline cellulose | 1.020 | mg |
| Water | 28.573 | mg |

*A 2% overage is included in these quantities to account for manufacturing losses.

### Prospective Example 5

A 7.5 mg amorphous sirolimus oral dosage tablet containing a 100 mg sugar overcoat is prepared according the procedure described in Example 1. The dispersion contained a 2:1 ratio of amorphous sirolimus:PLURONIC F68 (poloxamer 188), and is used at a concentration of 150 mg amorphous sirolimus/ml. The following lists the quantities of ingredients will be used.

Formula

| Ingredients* | Amount | |
|---|---|---|
| Amorphous Sirolimus | 7.5 | mg |
| PLURONIC F68 (poloxamer 188) | 3.75 | mg |
| Sucrose | 88.995 | mg |
| Povidone | 0.510 | mg |
| Microcrystaline cellulose | 1.020 | mg |
| Water | 15.398 | mg |

*A 2% overage is added to these quantities to account for manufacturing losses.

### Prospective Example 6

A 10 mg amorphous sirolimus oral dosage tablet containing a 100 mg sugar overcoat is prepared according the procedure described in Example 1. The dispersion contained a 2:1 ratio of amorphous sirolimus:PLURONIC F68 (poloxamer 188), and is used at a concentration of 150 mg amorphous sirolimus/ml. The following lists the quantities of ingredients will be used.

Formula

| Ingredients* | Amount |
|---|---|
| Amrophous Sirolimus | 10 mg |
| PLURONIC F68 (poloxamer 188) | 5 mg |
| Sucrose | 5.170 mg |
| Povidone | 0.510 mg |
| Microcrystaline cellulose | 1.020 mg |
| Water | 2.223 mg |

*A 2% overage is included in these quantities to account for manufacturing losses.

### IV Dosage Form Examples

### Prospective Example 7

Preparation of Sirolimus IV Concentration in Dimethylacetamide (50 mg/ml)

Rapamycin like compound IV Concentrate
in Dimethylacetamide (50 mg/ml)
Formula (Density - 0.944 g/ml):

| Ingredients | Amount |
|---|---|
| Amorphous Sirolimus @ 100% | 5.0 gm |
| Dimethylacetamide (DMA) qs | 100 ml or 94.4 gm |

Procedure:

1. Weigh the amorphous sirolimus into a suitable calibrated container.

2. Adjust volume to 100 ml with DMA.

3. Mix until a uniform solution results.

4. Sterile filter the solution.

5. Package into ampules and seal.

### Prospective Example 8

Preparation of Amorphouse Sirolimus IV Solution at 2.0 mg/ml

A. Diluent for amorphous Sirolimus IV at 2.0 mg/ml
Formula (Density - 1.081 gm/ml):

| Ingredients | Amount |
|---|---|
| Polysorbate 80, NF | 4.0 gm |
| Polyethylene Glycol 300, NF | 50.0 gm |
| Water for Injection, USP qs | 100 ml or 108.1 gm |

Procedure:

1. Weigh the Polysorbate 80 into a suitable calibrated container.

2. Add the Polyethylene Glycol 300 to the container in Step #1.

3. Adjust to final volume with Water for Injection, USP.

4. Mix until uniform.

5. Filter the resulting solution.

6. Fill 12.0 ml.+−.0.1 ml. into each 20 ml flint vial, seal and crimp.

7. Autoclave to achieve sterility:

,B. Amorphous Sirolimus IV solution at 2.0 mg/ml (constituted)
Formula (Density - 1.077 gm/ml):

| Ingredients | Amount |
|---|---|
| Amorphous Sirolimus IV Concentrate @ 50 mg/ml | 0.5 ml |
| Diluent for IV-Sirolimus | 12.0 ml |

Procedure:

1. Inject 0.5 ml of Amorphous Sirolimus IV Concentrate at 50 mg/ml into a vial container 12.0 ml of diluents for IV-Sirolimus using good sterile technique.

2. Shake until a clear solution results.

### Prospective Example 9

Preparation of Amorphous Sirolimus IV Solution at 4.0 mg/ml

A. Diluent for amorphous Sirolimus IV at 4.0 mg/ml
Formula (Density - 1.077 gm/ml):

| Ingredients | Amount |
|---|---|
| Polysorbate 80, NF | 8.0 gm |
| Polyethylene Glycol 300, NF | 50.0 gm |
| Water for Injection, USP qs | 100 ml or 107.7 gm |

Procedure:

1. Weigh the Polysorbate 80 into a suitably calibrated container.

2. Add the Polyethylene Gylcol 300 to the container in Step #1.

3. Adjust to final volume with Water for Injection, USP.

4. Mix until uniform.

5. Filter the resulting solution.

6. Fill 5.75 ml.+−.0.1 ml into each 10 ml flint vial, seal and crimp.

7. Autoclave to achieve sterility.

B. Amorphous Sirolimus IV solution at 4.0 mg/ml (constituted)
Formula (Density - 1.072 gm/ml):

| Ingredients | Amount |
|---|---|
| Amorphous Sirolimus IV Concentrate @ 50 mg/ml | 0.5 ml |
| Diluent for IV-Amorphous Sirolimus | 5.75 ml |

Procedure:

1. Inject 0.5 ml of Amorphous Sirolimus IV Concentration at 50 mg/ml into a vial container 5.75 ml of diluent for IV-Siroliums using good sterile technique.

2. Shake until a clear solution results.

### Prospective Example 10

The examples herein represent the batch production of ampules of sirolimus concentrate and vials of diluent for use in obtaining 0. 1, 0.5, 2.0 and 4.0 mg/mL. The sirolimus IV solutions may be constituted for injection in the same manner as in Examples 2B and 3B.

A. SirolimusIV Concentrate 50 mg/ml

| | | | Representative |
|---|---|---|---|
| | | Input/ | Batch Formula |
| | Claim/mL | Ampule | 10,000 Ampules |
| Active Ingredient | | | |
| Amorphous Rapamycin @ | 0.050 g | 0.0325 g | 0.325 kg |
| 100% | | | |
| Inactive Ingredients: | | | |
| Dimethyl acetamide qs ad | | 0.65 mL or | 6.50 L or |
| | | 0.61 g | 6.14 kg |
| Density = 0.944 g/mL | | | |

| | | |
|---|---|---|
| | | Representative |
| | | Batch Formula |
| Active Ingredient | Input/Vial | 10,000 Vials |

B. Diluent for Sirolimus IV at 0.1 mg/mL

| | | |
|---|---|---|
| Polysorbate 80, NF | 4.00 g | 40.0 kg |
| Polyethylene Glycol 300, NF | 50.0 g | 500 kg |
| Water for Injection, USP qs ad | 100 mL or | 1000 L or |
| | 108 g | 1081 kg |
| Density - 1.081 g/mL | | |

C. Diluent for Rapamycin IV at 0.5 mg/mL

| | | |
|---|---|---|
| Polysorbate 80, NF | 2.00 g | 20.0 kg |
| Polyethylene Glycol 300, NF | 25.0 g | 250 kg |
| Water for Injection, USP qs ad | 50.0 mL or | 500 L or |
| | 54.1 g | 541 kg |
| Density - 1.081 g/mL | | |

D. Diluent for Rapamycin IV at 2 mg/mL

| | | |
|---|---|---|
| Polysorbate 80, NF | 0.480 g | 4.80 kg |
| Polyethylene Glycol 300, NF | 6.00 g | 60.0 kg |
| Water for Injection, USP qs ad | 12.0 mL or | 120 L or |
| | 13.0 g | 130 kg |
| Density - 1.081 g/mL | | |

E. Diluent Rapamycin IV at 4 mg/mL

| | | |
|---|---|---|
| Polysorbate 80, NF | 0.460 g | 4.60 kg |
| Polyethylene Glycol 300, NF | 2.88 g | 28.8 kg |
| Water for Injection, USP qs ad | 5.75 mL or | 57.5 L or |
| | 6.19 g | 61.9 kg |
| Density - 1.077 g/mL | | |

Note:
A-E If the potency of sirolimus is less than 100%, the input must be adjusted to give claim potency.

Procedures for preparations A-E.

A. Sirolimus IV Concentrate at 50 mg/ml Procedure:

1. Weigh the amorphous sirolimus into a suitable calibrated container.

2. Add dimethylacetamide to achieve the appropriate volume or weight

3. Mix until a solution results.

4. Maintain sterile conditions throughout filtering, filling and sealing.

5. Filter the solution from Step #3 through a 0.2 micron filter.

6. Fill 0.65 ml.±.0.05 ml (0.61 g+0.05 g) of the solution from Step #5 into each 1 ml amber ampule and seal.

7. Store under refrigeration.

B. Sirolimus IV Diluent at 0.1 mg/ml Procedure:

1. Weigh the Polysorbate 80 into a suitable container.

2. Add the appropriate weight of the Polyethylene Glycol 300 to the container in Step #1.

3. Add Water for Injection to achieve the appropriate volume or weight.

4. Mix until a solution results.

5. Filter the solution from Step #4 through a 0.2 micron filter.

6. Fill 100 mL.±.2 mL (108 g.±.2.2 g) of the solution from Step #5 into each 100 mL flint vial, seal with a barrier faced stopper and crimp with an aluminum seal.

7. Sterilize by steam autoclave.

8. Store at room temperature or under refrigeration.

C. Sirolimus IV Diluent at 0.5 mg/ml Procedure:

1. Weigh the Polysorbate 80 into a suitable container.

2. Add the appropriate weight of the Polyethylene Glycol 300 to the container in Step #1.

3. Add Water for Injection to achieve the appropriate volume or weight.

4. Mix until a solution results.

5. Filter the solution from Step #4 through a 0.2 micron filter.

6. Fill 50 mL.±.1 mL (54 g.±.1.1 g) of the solution from Step #5 into each 100 mL flint vial, seal with a barrier faced stopper and crimp with an aluminum seal.

7. Sterilize by steam autoclave.

8. Store at room temperature or under refrigeration.

D. Sirolimus IV Diluent at 2 mg/ml Procedure:

1. Weigh the Polysorbate 80 into a suitable container.

2. Add the appropriate weight of the Polyethylene Glycol 300 to the container in Step #1.

3. Add Water for Injection to achieve the appropriate volume or weight.

4. Mix until a solution results.

5. Filter the solution from Step #4 through a 0.2 micron filter.

6. Fill 12.0 mL.±.0.1 mL (13.0 g.±.0.1 g) of the solution from Step #5 into each 20 mL flint vial, seal with a barrier faced stopper and crimp with an aluminum seal.

7. Sterilize by steam autoclave.

8. Store at room temperature or under refrigeration.

E. Sirolimus IV Diluent at 4 mg/ml Procedure:

1. Weigh the Polysorbate 80 into a suitable container.

2. Add the appropriate weight of the Polyethylene Glycol 300 to the container in Step #1.

3. Add Water for Injection to achieve the appropriate volume or weight.

4. Mix until a solution results.

5. Filter the solution from Step #4 through a 0.2 micron filter.

6. Fill 5.75 mL.±.0.1 mL (6.2 g.±.0.1 g) of the solution from Step #5 into each 10 mL flint vial, seal with a barrier faced stopper and crimp with an aluminum seal.

7. Sterilize by steam autoclave.

8. Store at room temperature or under refrigeration.

### Oral Liquid Dosage Form

### Prospective Example 11

Sirolimus Oral at 1 mg/ml

A sirolimus oral formulation at a concentration of 1 mg/ml can be formulated for the following active and inactive ingredients by the procedural steps which follow:

| | | | | Batch Formula | |
|---|---|---|---|---|---|
| | | | | 10,000 | |
| | Conc. | Input | | bottles | |
| Active Ingredient: | | | | | |
| Amorphous sirolimus @ | 1.00 mg/ml | 0.025 | g | 0.250 | kg |
| 100% | | | | | |
| Inactive Ingredients: | | | | | |
| Polysorbate 80, NF | 10.8 mg/ml | 0.270 | g | 2.700 | kg |
| Phosal 50 PG.RTM | 1.00 ml or | 25.0 | ml | 250.0 | L |
| propylene glycol | 1.005 gm | 25.125 | g | 251.25 | kg |
| and lecithin q.s. ad | | | | | |

Density of the Final Formulation 1.005 g/ml

If the potency of the amorphous sirolimus is less than 100%, the input must be adjusted to achieve the claimed potency.

Method of Manufacture

Procedure:

1. Weigh the amorphous sirolimus into a suitable container.

2. Add the Polysorbate 80 to the container in step #1

3. Adjust to the final volume with Phosal 50 PG.

4. Mix until the amorphous sirolimus is dissolved.

5. Fill 25 ml.±.1.25 ml (25.125 g.±.1.256 g) into each one ounce amber glass bottle. It is preferable to seal with a child resistant cap.

For improved wettability and ease of solution, an alternative order of addition of the ingredients and amounts presented above is as follows:

1. Polysorbate 80.

2. A portion of the Phosal 50 PG propylene glycol and lecithin.

3. Amorphous Sirolimus.

4. The remaining Phosal 50 PG propylene glycol and lecithin. The amorphous sirolimus in these formulations may also be comminuted by use of a mill or mortar and pestle and passed through an 80 mesh screen.

### Prospective Example 12

Sirolimus Oral at 5 mg/ml

A sirolimus oral formulation at a concentration of 5 mg/ml can be formulated from the following active and inactive ingredients by the procedural steps which follow:

| | | | Batch |
|---|---|---|---|
| | | | Formula |
| | | | 10,000 |
| | Conc. | Input | bottles |
| Active Ingredient: | | | |
| Amorphous Sirolimus @ 100% | 5.00 mg | 0.125 g | 1.250 kg |
| Inactive | | | |
| Ingredients: | | | |
| Polysorbate 80, NF | 10.8 mg | 0.270 g | 2.70 kg |
| Phosal 50 PG | 1.00 ml | 25.0 ml | 250.0 L or |
| propylene glycol | or 1.005 gm | or 25.125 g | 251.25 kg |
| and lecithin q.s. ad | | | |

Density of the Final Formulation 1.005 g/ml.

If the potency of the amorphous sirolimus is less than 100%, the input must be adjusted to give the claimed potency.

The procedural steps for formulation and storage of the 5 mg/ml oral sirolimus formulation are the same as those listed in Example 1, as are the alternative order of addition of ingredients and the methods of comminution.

### Prospect Example 13

The formulation of this Example 13 can be produced using the ingredients which follow and the methods indicated below:

| Ingredients | Amount |
|---|---|
| Amorphous Sirolimus @ 100% up to | 1.0 gm |
| Polysorbate 80, NF | 1.0 ml or 1.08 gm |
| Phosal 50 PG lecithin and propylene glycol | 100 ml or 100.5 gm |
| q.s. | |

Method of Formulation

1. Weigh the amorphous rapamycin into a suitable container.

2. Add the Polysorbate 80 into the container of Step #1.

3. Adjust to the final volume with Phosal 50 PG.RTM. propylene glycol and lecithin.

4. Mix until a solution results.

Alternatively, this formula can be packaged in a suitable container or encapsulated into a capsule.

### Prospective Example 14

| Formula | Ingredients |
|---|---|
| Amorpous Rapamycin @ 100% up to | 2.5 grams |
| Polysorbate 80, NF | 5.0 ml or 5.4 gm |
| Absolute Ethanol | 12.67 ml or 10.0 gm |
| Phosal 50 PG lecithin and propylene glycol | 100 ml |
| q.s. | |

This formulation can be produced by the following steps:

1. Weigh the amorphous rapamycin into a suitable container

2. Add the absolute ethanol to the container in Step #1. Mix until dissolved.

3. Add the polysorbate 80 to the container in Step #2. Mix until uniform.

4. Add Phosal 50 PG lecithin and propylene glycol to adjust to the final volume.

5. Mix until uniform.

Alternatively, this formula can be packaged in a suitable container or encapsulated into a capsule.

### Prospective Example 15

The oral formulations of this invention, such as those disclosed above, may also be prepared in encapsulated forms, such as formulations within starch or SEG capsules.

The following procedure describes a method which may be utilized to prepare such encapsulated formulations.

Procedure:

1) Add to a container, NF, the Polysorbate 80.

2) Add to the Polysorbate 80 of Step #1 80% of the the required Phosal 50 PG.

3) Weigh the amorphous sirolimus component of the formulation into the container of Step #2.

4) Adjust to the final formulation weight with Phosal 50 PG.

5) Establish a nitrogen atmosphere over the formulation and maintain until the capsules are filled.

6) Mix the formulation until the amorphous sirolmus is dissolved.

7) Pass the formulation solution through a particulate (such as a 100 mesh screen) or scintered glass filter.

8) Fill 0.50 ml of the Step #7 material into capsule shells using an automatic syringe dispensing unit and seal the capsule.

9) Package the filled capsules upon completion of encapsulation. An example of a preferred package is a conventional blister package with a perforable metal foil backing.

10) Optionally store the finished encapsulated product at refrigerated conditions (2°-8° C.) protected from light.

The primary capsule sealant for the starch capsule may be a 5% Dextrin, NF, aqueous solution. It is preferable to heat purified water to 50°-60° C. prior to compounding to facilitate dissolution of the Dextrin. Prior to use it is also preferable to filter the Dextrin solution through a suitable particulate filter.

### Prospective Example 16

Bioavailability

The bioavailability of any of the formulation provided above or in the specification may be determined by methods known in the art. Suitable methods for testing such bioavailability include but are not limited to:

a) Testing the formulation in cynomolgus monkeys. Cynomolgus monkeys may be administered the formulations provided above, at appropriate doses and the serum concentrations may be determined over time after dosing to determine the optimum dosage profile:

b) Formulations containing an amorphous rapamycin like compound at appropriate concentrations, prepared as described above, may be administered to healthy male human volunteers between the ages of 18 and 45, from whom blood samples were drawn at the time intervals table below. The sirolimus blood samples may be assayed for whole blood sirolimus concentration using a validated (ESP)-HPLC-MS method.

One appropriate example of time intervals to test blood concentrations would be as follows:

| Time Interval Following | Blood Concentration |
|---|---|
| Administration (Hours) | (conc. = ng/ml) |
| 0.33 | |
| 0.67 | |
| 1 | |
| 2 | |
| 3 | |
| 4 | |
| 5 | |
| 8 | |
| 12 | |
| 18 | |
| 24 | |
| 48 | |

1. A pharmaceutical dosage form comprising substantially amorphous rapamycin like compounds and a pharmaceutically acceptable excipient.

2. The pharmaceutical dosage form of claim 1 wherein the substantially amorphous rapamycin like compound is sirolimus.

3. The pharmaceutical dosage form of claim 2 wherein the substantially amorphous sirolimus contains less than 30 weight percent crystalline sirolimus.

4. The pharmaceutical dosage form of claim 2 wherein the substantially amorphous sirolimus contains less than 10 weight percent crystalline sirolimus.

5. The pharmaceutical dosage form of claim 2 wherein the substantially amorphous sirolimus contains less than 5 weight percent crystalline sirolimus.

6. The pharmaceutical dosage form of claim 2 wherein the substantially amorphous sirolimus contains less than 1 weight percent crystalline sirolimus.

7. The pharmaceutical dosage form of claim 2 wherein per unit dose the pharmaceutical dosage form contains from about 0.1 mg to about 2 mg of the sirolimus.

8. The pharmaceutical dosage form of claim 2 per unit dose the pharmaceutical dosage form contains from about 0.5 mg to about 1 mg of the sirolimus.

9. The pharmaceutical dosage form of claim 1 where the pharmaceutical dosage form is a solid dosage form.

10. The pharmaceutical dosage form of claim 7 wherein the solid dosage form is selected from the group consisting of tablets, capsules, caplets, gelcaps, geltabs, powders and granules.

11. The pharmaceutical dosage form of claim 8 wherein the solid dosage form is selected from the group consisting of tablets, capsules, gelcaps and geltabs.

12. The pharmaceutical dosage form of claim 11 wherein the rapamycin like compound is sirolimus.

13. The pharmaceutical dosage form of claim 1 wherein the dosage form is an oral dosage form.

14. The pharmaceutical dosage form of claim 13 wherein the rapamycin like compound is sirolimus.

15. The pharmaceutical dosage form of claim 1 wherin the dosage form is an injectable dosage form.

16. The pharmaceutical dosage form of claim 15 wherein the like compound is sirolimus.

17. The pharmaceutical dosage form of claim 1 wherein the pharmaceutical dosage form is a suspension containing amorphous rapamycin.

18. A process for making a pharmaceutical dosage form comprising admixing a substantially amorphous rapamycin like compound with at least one pharmaceutically acceptable excipient.

19. The pharmaceutical dosage form of claim 18 wherein the like compound is sirolimus.
